(19) United States
(12) Patent Application Publication
(10) Pub. No.: US 2002/0168429 A1
(43) Pub. Date: Nov. 14, 2002

(54) Nutritional supplement and method of delivery

(76) Inventor: Douglas G. Mann, Venice, FL (US)

Correspondence Address: Charles S. Sara, DEWITT ROSS & STEVENS, S.C., Suite 401, 8000 Excelsior Drive, Madison, WI, 53717-1914, US

(21) Appl. No.: 10/127,214

(22) Filed: Apr. 22, 2002

Related U.S. Application Data
(63) Continuation-in-part of application No. 09/303,808, filed on Apr. 30, 1999, now Pat. No. 6,231,866.
(63) Continuation-in-part of application No. 09/789,797, filed on Feb. 20, 2001, now Pat. No. 6,440,467.
(60) Provisional application No. 60/083,566, filed on Apr. 30, 1998.

(51) Int. Cl. A61K 35/78

(52) U.S. Cl. 424/732

(57) ABSTRACT

A method of delivering dietary supplements, in the form of intact anthocyanins, into the lower gastrointestinal tract of a body for absorption from an aqueous medium comprising: expressing juice from one or more fruits, which fruits include anthocyanins, thereby yielding a juice portion and a pomace portion; concentrating the juice portion to yield a juice concentrate; mixing the juice concentrate with the pomace portion; drying the juice-infused pomace to yield a free-flowing, non-hygroscopic powder formulation to yield the dietary supplement; and orally ingesting the dietary supplement in the form of capsules, tablets, shakes, drinks, energy supplements, energy bars, and the like.

### CROSS-REFERENCE TO RELATED APPLICATIONS

This application claims priority under 35 U.S.C. §119(e) to provisional U.S. patent application Ser. No. 60/083,566, filed Apr. 30, 1998, abandoned in favor of U.S. patent application Ser. No. 09/303,808, filed Apr. 30, 1999 and recently issued on May 15, 2001 as U.S. Pat. No. 6,231,866, of which is incorporated by reference herein.

This application is a continuation-in-part of U. S. patent application Ser. No. 09/789,797 filed Feb. 20, 2001, in the name of Douglas G. Mann and entitled INFUSED VEGETABLE, HERB AND/OR SEED FIBER PRODUCT AND DIETARY SUPPLEMENTS CONTAINING SAME, which is incorporated herein by reference in its entirety.

### REFERENCE TO CITATIONS

Full bibliographic citations of the references cited in this document are found in the bibliographic section preceding the claims.

### FIELD OF THE INVENTION

The invention is directed to: (i) a method of producing a reconstituted vegetable, fruit, herb, and/or seed product, the product produced using the method, and dietary supplements containing the product; (ii) other methods which provide all natural solutions for carrying and delivering nutraceutical supplements into the human body; (iii) a unique cranberry nutraceutical product which can be used effectively to promote and maintain a healthy urinary tract; and (iv) a method of delivery for a nutraceutical compound.

### DESCRIPTION OF THE PRIOR ART

One object of nutraceutical dietary supplements is to deliver the active components of the nutraceutical into the human body in an efficacious basis. However, many of these supplements after oral ingestion are substantially degraded by stomach acids before they can deliver their payload to the intestine for assimilation into the blood stream. For example, most of the cranberry powdered ingredients being currently employed in dietary supplements dissolve quickly in the stomach and thus have limited bioavailability. While there are various drug delivery systems used in the pharmaceutical industry to increase efficacy, there is an absence of methods available for natural delivery of nutraceutical products.

There is also a need to create a nutritional supplement which allows for the effective delivery of anthocyanins to the body. Anthocyanins are the glycosolated (sugar-bound) analogs of anthocyanidins. The anthocyanidins are purportedly the species that are actually active in the body at the site of action. A study by Youdim, et al. (2000) investigated the potential antioxidant properties of blueberry polyphenolics in vitro and in vivo, using red blood cell (RBC) resistance to reactive oxygen species (ROS) as the model. In vitro incubation with anthocyanins or hydroxycinnamic acids (HCA) was found to enhance significantly RBC resistance to peroxide-induced ROS production. This protection was also observed in vivo following oral supplementation to rats. However, only anthocyanins were found to afford protection at a significant level, this at 6 and 24 hours post supplementation. This protection was consistent with the measured plasma levels of anthocyanins. The difference in absorption between anthocyanins and HCA is likely to have contributed to the observed difference in their abilities to afford protection to RBC. This protection represents a positive role following dietary consumption of intact anthocyanins allowing for conversion to anthocyanidins in vivo; however as the anthocyanidins are insoluble in water, they therefore cannot be easily absorbed by the body if ingested in their active non-glycosolated state. Thus it is necessary to absorb anthocyanins and allow the body to convert these to the anthocyanidins at the site of use.

The anthocyanins possessing the bound sugar renders them water soluble and thus easily absorbed by the body. This has been demonstrated by Tsuda, et al. (1999). In order to clarify the mechanism of action of cyanidin 3-O-beta -D-glucoside, they investigated the absorption and metabolism of cyanidin 3-O-beta -D-glucoside in rats. Cyanidin 3-O-beta -D-glucoside appeared in the plasma after the oral administration of enterically coated cyanidin 3-O-beta -D-glucoside. Protocatechuic acid, which is produced by the degradation of a glycon of cyanidin 3-O-beta -D-glucoside (cyanidine), and was not present in the plasma. A similar study by the same authors has further clarified how cyanidin 3-O-beta-D- glucoside is absorbed and metabolized in vivo. Tsuda, et al. (2000). Although cyanidin 3-O-beta -D-glucoside rapidly appeared in the plasma, cyanidin was not detected, although it was present in the jejunum. It should be noted, however, that the chemical linkage between the anthocyanidin and the sugar group is highly fragile in acid environments. Thus, when intact anthocyanins are ingested orally, they are rapidly degraded by the acidic environment of the upper gastrointestinal tract to the sugar group and the now insoluble, unabsorbable anthocyanidin.

It should be noted, however, that the chemical linkage between the anthocyanidin and the sugar group is highly fragile in acid environments. Thus, when intact anthocyanins are ingested orally, they are rapidly degraded by the acidic environment of the upper gastrointestinal tract to the sugar group and the now insoluble, unabsorbable anthocyanidin.

Thus, there is a clear need for an all-natural edible delivery method for anthocyanins and other nutraceutical compounds that can withstand highly acidic environments.

### SUMMARY OF THE INVENTION

The present invention is directed to a method of orally delivering nutraceutical products into the gastrointestinal tract of an animal for absorption, comprising (1) expressing the juice from one or more plant materials comprising nutraceutical products to yield a juice portion and a pomace portion, (2) concentrating the juice portion to form a juice concentrate, (3) infusing the concentrate with the pomace portion, whereby the concentrate is absorbed into the pomace, and (4) orally delivering the product of step d to the animal.

In particular, the present invention is directed to an all natural method of orally delivering nutraceutical products, which has been given the name BIO-SHIELD, and which term shall be used to designate this method. The key benefit of the BIO-SHIELD matrix is to maintain the integrity of the anthocyanidin to glucose bond thereby allowing intact anthocyanins to be passed into the lower gastrointestinal tract where they may be readily absorbed from an aqueous medium.

In yet another respect, the invention concerns an all natural unique method of orally delivering nutraceutical compositions into the human body in such a manner that the bio-active compounds contained therein are more effectively absorbed and utilized in the human body.

In another respect, the invention pertains to an all natural method for producing powdered ingredients which are non-hygroscopic and which have enhanced flow characteristics without the use of unnatural excipients.

In yet another respect, the invention pertains to a cranberry-based nutraceutical composition containing active components which inhibit the adhesion of bacteria to surfaces in the urinary tract and which assists in the promotion and maintenance of a healthy urinary tract.

In yet another respect, the invention pertains to a method for producing a cranberry-based nutraceutical drink, such as tea or a single-serve juice functional beverage. These liquid could also be frozen to make an appealing nutraceutical popsicle for children.

A second embodiment of the invention is directed to a dietary supplement alternatively produced by infusing pomace with oil or other extract derived from the same or a different plant and drying the infused fibers. In a first step, the oil or other extract is expressed from plant material of plants, thereby yielding an oil or extract portion and a pomace portion. The oil or extract portion is then infused with the pomace portion, whereby the oil or extract is absorbed into the pomace. The pomace so treated is then dried and milled, and optionally tableted or placed in soft or hard capsules.

A third embodiment of the invention is directed to a highly concentrated, unpurified nutritional dietary fiber product produced from pomace derived from the same or different plant and made into a powdered form. In a first step, juice is expressed from plants, thereby yielding a juice or oil portion and a pomace portion. The juice or oil portion is discarded. The pomace is then dried, milled and packaged.

A fourth embodiment of the invention is drawn to the product produced using the process described immediately above.

In particular, the present invention is directed to an all natural method of orally delivering nutraceutical products, which has been given the name BIO-SHIELD, and which term shall be used to designate this method. The key benefit of the BIO-SHIELD matrix is that it protects against digestive enzyme attack. By this, it is meant that the matrix acts to maintain the integrity of the anthocyanidin-to-glucose bond thereby allowing intact anthocyanins to be passed into the lower gastrointestinal tract where they may be readily absorbed from an aqueous medium, thus delivering a sustained release of bioactive components with minimum degradation. The BIO-SHIELD matrix also provides time release of active ingredients to sites of action.

The BIO-SHIELD matrix, being composed of natural plant fibers, is considerably more tolerant to acid environments, such as stomach acids. Under acidic conditions, the BIO-SHIELD matrix offers protection from surrounding acids against degradation of anthocyanins, as it would in nature. Without wishing to be held to one theory, it is believed that this effect is due to a contraction of lignin-cellulose fibers in the presence of acids, thereby decreasing the permeability of the matrix to the aqueous acidic environment, and thus limiting the degradation of the anthocyanins that have been absorbed onto the BIO-SHIELD matrix.

Once in the duodenum, the environment surrounding this BIO-SHIELD matrix returns to a neutral state, the BIO-SHIELD matrix relaxes and allows absorbed anthocyanins to dissolve into the aqueous environment. The protected anthocyanins can be shown to exist in an unmodified state by experiments similar to those carried out by Lapidot (1998), whereby they determined the potential bioavailability, in human, of several anthocyanins from red wine.

The BIO-SHIELD matrix can be considered as a natural enteric coating that can be used to facilitate the absorption of intact bioavailable anthocyanins, and further to prolong the stability of anthocyanins. The natural matrix structure of the BIO-SHIELD product could be considered as a buffer to acidic environments, as would be in the case of the natural fruit from where it was derived, and thus limits the rate of natural degradation of the anthocyanins.

The BIO-SHIELD product, being composed of natural plant fibers, is considerably more tolerant to acid environments, especially since the BIO-SHIELD matrix is preferably made from cranberries. Under acidic conditions the BIO-SHIELD matrix offers protection from surrounding acids against degradation of anthocyanins, as it would in nature. Without wishing to be held to one rationale, it is believed that this effect is due to a contraction of lignin-cellulose fibers in the presence of acids, thereby decreasing the permeability of the matrix to the aqueous acidic environment, and thus limiting the degradation of the anthocyanins that have been absorbed onto the BIO-SHIELD matrix.

Once in the duodenum, the environment surrounding this BIO-SHIELD matrix returns to a neutral state, the BIO-SHIELD matrix can relax and allow absorbed anthocyanins to dissolve into the aqueous environment. The protected anthocyanins can be shown to exist in an unmodified state by experiments similar to those carried out by Lapidot (1998), whereby they determined the potential bioavailability, in human, of several anthocyanins from red wine.

The BIO-SHIELD matrix can be considered as a natural enteric coating that can be used to facilitate the absorption of intact bioavailable anthocyanins, and further to prolong the stability of anthocyanins. Fossen (1998). The natural matrix structure of the BIO-SHIELD product could be considered as a buffer to acidic environments, as would be in the case of the natural fruit from where it was derived, and thus limits the rate of natural degradation of the anthocyanins.

The invention is an edible 100% plant matter composition which can be used as a nutritional ingredient in place and instead of other highly concentrated, unpurified fiber products such as bran, gum or psyllium-seed husk. It can also be presented in unit dosage form to promote and maintain a healthy life. In the case where cranberries are utilized as the plant material source, the composition is a cranberry fiber products containing some of the bio-active values of cranberries.

In addition to its clear health benefits, another distinct advantage of the present invention is that it that it utilizes plant-derived pomace, which would otherwise be discarded as waste. For example, when cranberry juice concentrate is added to the cranberry pomace, the resulting reconstituted cranberry product makes an excellent nutritional supplement which does not require nutritionally insignificant excipients such as sweeteners, desiccants, binding agents, silicon dioxide, tricalcium phosphate and the like.

Further benefits of the present invention are manifest. By reconstituting concentrated juice with the natural fibers, the nutritionally- and pharmacologically-active ingredients present in the vegetable, fruit, herb, or seed are concentrated in comparison to their concentrations as found in nature. Consequently, the bio-active compounds found naturally in the plants are fortified. Also, on a per weight basis, the resultant product is far richer in beneficial vitamins, anthocyanins, proanthocyanins, antioxidants, and other beneficial components.

Moreover, the process and the resultant product utilize the entire natural source, including the vegetable, fruit, skin, seeds, and fibrous portions thereof, and not simply an extract of the natural plant source.

In essence, the process yields a powdered version of an entire vegetable, fruit, herb, or seed. The resultant product contains the complete complement (juice, skin, seeds, fiber) of the source vegetable matter, not just the juice portion. By utilizing only low-temperature processing, the resultant product preserves the natural enzyme activities found in the fresh vegetable, fruit, herb or seed. In its preferred form, the process does not require any unnatural substances; hence the finished product does not contain any unnatural substances. However, it is within the scope of the present invention to add other ingredients if desired.

Additionally, the final product is capable of being finely milled and can therefore easily be formulated into any number of unit dosage forms, such as tablets or capsules. The product need not be refrigerated and is storage stable for at least a period of months, if not years. This makes formulation, storage, and transport of the product extremely attractive.

When formulated into unit dosages, such as tablets or capsules, the product of the invention is easily delivered orally. Because the bio-active ingredients are infused into a generally fiber matrix, the bio-active components are shielded from degradation during transit through the stomach, thereby delivering a maximum concentration of bio-active ingredients in the intestines. The natural pectin components of the product slow down the digestive process in the intestines and provide a natural sustained release of the active compounds from the fiber matrix, thereby enhancing the bioavailability of the active compounds. The insoluble fiber portion, while indigestible, serves as a bulking agent to promote regularity and good intestinal health and functioning.

Further advantages of the invention will appear from a complete reading of the Detailed Description, below.

### DETAILED DESCRIPTION OF THE INVENTION

An all natural method of orally delivering nutraceutical products to the body system, has been given the name BIO-SHIELD, which term shall be used to designate the method described below. The BIO-SHIELD method comprises using a composition produced using the method described below and placing the composition into a capsule, tablet, softgel, liquid drink, or nutraceutical energy bar and then orally ingesting it.

Because the bio-active ingredients are infused into a fiber matrix, the bio-active components are shielded from degradation during transit through the stomach, thereby delivering a maximum concentration of bio-active ingredients into the intestine. The natural pectin components of the fiber matrix slow down the digestive process in the intestines and provide a sustained release of the active compounds from the fiber matrix, thereby enhancing the bioavailability of the active compounds.

A first embodiment of the invention is directed to a dietary supplement produced by infusing plant-derived fiber with juice concentrate derived from the same or different plant and drying the infused fibers. The term “plant-derived fiber” is also sometimes referred to in this and other publications as “pomace,” “marc,” and “press cake.” For purposes of this application, the plant-derived fiber portion will be referred to as pomace. In a first step, juice is expressed from plant material of fruits, vegetables, herbs, spices, etc. (hereinafter referred to collectively as plants) and seeds of plants, thereby yielding a juice portion and a pomace portion. The juice portion is concentrated to yield a juice concentrate, and the juice concentrate is then infused with the pomace portion, whereby the concentrate is absorbed into the pomace. The pomace so treated is then dried and milled and optionally tableted or capsulated.

The two principal components of the BIO-SHIELD system are the natural fruit fibers and pectins (pomace) combined with the bio-active ingredient.

Pomace

The BIO-SHIELD matrix can be produced with virtually any plant material, including whole plants, whole fruits, whole vegetables, spices, herbs, seeds, skin, bark, leaves, roots, tubers, or parts thereof, may be used in the present invention. Preferably, an entire fruit or vegetable or an entire plant be used, although this is not required. The preferred plant materials to be utilized in the invention fall into two categories: Category I: whole cranberries, blueberries, bilberries, elderberries, aronia, squash, carrots, apples and raspberries; and Category II: Nigella sativa, saw palmetto, cranberry seed oil, flax seed oil, borage oil, alfalfa, and Echinacea. Preferably, each of these materials is formulated separately to yield a powdered product derived from a single plant source. However, if desired, mixtures of various plant materials may be commingled and processed simultaneously.

The preferred composition is 100% cranberry. Cranberry fiber contains an abundance of pectin, and has a great deal of carrying capacity for protection of bio-active ingredients. The cranberry fiber has a capacity to absorb 3-5 times its weight in bio-active ingredients, and it can also absorb a much greater amount of oil than the pomace of most other natural plants or fruits. As a result, cranberry fiber is an excellent pomace for transporting not only cranberry concentrates, but other fruit and herb concentrates and oils, as well, such as blueberries, bilberries, elderberries, saw palmetto, as well as a wide range of other bioactive oils.

The preferred plant fiber is the pomace taken directly from the presses used to express the juice, oil or other liquid extract from the plant material. However, it is within the scope of the invention to mix the pomaces and juices. For example, there may be value in infusing the concentrated juice of cranberries into the pomace derived from apples, blueberries, carrots, squash or other plants. The pomace need not be dried prior to its use in the invention.

However, the final fiber matrix can have a portion of other natural fibers mixed in, for example blueberry, bilberry, saw palmetto fiber, or the fibers of other plants. At this point, different concentrates of oil, juice, or bio-active extracts are added into the fiber.

Bio-active Ingredient

The bio-active ingredient can include any of a number of nutraceutically, pharmaceutically or medicinally beneficial products or drugs which are ingested in the human or other animal body system. As discussed above, most of these products are sensitive to degradation by stomach acids, enzymes and the like before they are transported to the target organ, such as the intestine. By enveloping the bio-active ingredient in a fiber matrix “wrapper,” the bio-activity of the ingredient can be sustained as it passes through the stomach.

Reference is made above to juice concentrates, which contain bio-active ingredients. For example, many juices contain beneficial anthocyanidins, again described above. By pressing out the juice from the fruit or vegetable and concentrating it as described above, the bulk of the liquid is removed but the beneficial ingredients remain intact. As will be described in the following paragraphs, the concentrated liquid is absorbed with the now-processed pomace to form the BIO-SHIELD product.

It is within the scope of the present invention to add additional supplements and drugs, such as vitamins, minerals, and antibiotics to the concentrated juice or oils prior to re-infusion with the pomace.

In use, the composition, encapsulated or not, is ingested orally as a dietary supplement to promote the general health of the user. The composition can also be used as a food additive for fortification of fiber bars, cereals, breads, and drinks.

### Steps For Producing BIO-SHIELD Product

Expressing Juice

As with the process for producing the CRAN-MAX product described above and in U.S. Pat. No. 6,231,866, the first step in producing the BIO-SHIELD product is to express the juice from the fruit or vegetable. This is accomplished by any of a number of known processes again as described above and in U.S. Pat. No. 6,231,866.

Concentrating the Juice

The expressed juice is then concentrated as described above in the CRAN-MAX process. If desired, the concentrated juice can be supplemented with vitamins, minerals, drugs, or other ingredients. Proper proportions of ingredients are measured, added to the juice concentrate and blended continuously while being placed into the BIO-SHIELD material. The proper portion of wet formulation is then blended with the proper portion of BIO-SHELD material until evenly and thoroughly absorbed.

Drying the Pomace

The remaining pomace, after the juice has been expressed, is collected, packaged, and frozen within less than a 24-hour period, but preferably within a 6-hour period. The pomace is then pressed and dried in a manner similar to that described above with respect to the CRAN-MAX product. Preferably, the pomace is dried to a moisture content between about 3% and 6% moisture content, and most preferably to 5% moisture.

Mixing the Concentrate and the Pomace

The juice concentrate and the pomace are preferably mixed at a ratio ranging from between about 1:1 (juice concentrate to pomace) to 1:4 (wt/wt) based upon a 50 brix juice concentrate and the calculated dry weight of the pomace. The pomace and concentrated juice are then combined in a batching vessel along with an amount of guar gum for binding purposes. Additional nutritional and/or nutraceutical substances from the group consisting of vitamins, minerals, herbs, and the like, may be added during the mixing stage. The ratio of juice concentrate to pomace is established prior to the addition of any further ingredients.

The juice and pomace and any additives are mixed thoroughly to ensure that the entire bulk of the pomace is contacted by the concentrated juice. Preferably, this mixing is done at a temperature between about 40□F and 75□F. The mixture is allowed to steep for up to 24 hours to allow the liquid to be fully absorbed into the pomace.

Drying the Pomace/Concentrate Mixture

The pomace/concentrate mixture is then dried. This can be done on drying racks in a conventional dehydrator or by vacuum drying means, or by any other means for drying known to the art of food and pharmaceutical processing. Low-temperature drying means (not to exceed about 140□F.) are greatly preferred. It is preferred that the moisture content of the dried mixture be no more than about 3% by weight.

Deseeding

As described in the parent application, all seeds are then preferably removed from the pomace. The seeds are not necessary to end fiber product, and may deter the absorption capability of the fiber matrix. In addition, the seeds have a nutraceutical value by themselves and can be used in other processes. The deseeding process is accomplished by the methods described in the parent application.

Milling

The product is then milled to a uniform size if desired. Generally, milling to a mesh size of between about 50 and about 80 yields a product which readily flows and can easily be packaged, transported, and formulated into dosage form (if desired). A 50-80 mesh powder is easily pelletized or capsulated using suitable and conventional machinery.

Encapsulation (Soft or Hard Shelled) or Tabletine

The final product can then be prepared for consumption by encapsulation, tableting, or combining with drug, food, or drink products as described above.

It is also within the scope of the present invention to mix fibers, for example cranberry fibers with blueberry fibers, and then add blueberry concentrate to develop a product known as BLUE-MAX. In this manner, the blueberry bio-active ingredients, which are the anthocyanins, will be delivered into the blood stream and protected through the stomach. An added advantage here is that the natural color of the product is blue, suggesting blueberries.

The same process can work for saw palmetto oil, cranberry seed oil, flax seed oil, or other essential oils. For example, saw palmetto fiber typically can only hold 12% oil. To enhance this value, saw palmetto fiber can be combined with a requisite amount of cranberry fiber to achieve up to 25% oil carrying capacity on an all natural basis. If desired, other berry matrices, such as blueberry and bilberry fiber matrices can be combined to deliver a bio-active ingredient depending of the situation.

Nutraceutical Drink or Food

Another aspect of the invention is a nutraceutical drink, shake, or energy bar, which are made by expressing juice from one or more fruits and concentrating this juice to yield a juice concentrate. This juice concentrate is then mixed with the pomace portion of the fruit, as well as additional nutritional and edible stabilizing compounds to produce a juice infused pomace drink, shake, or energy bar product.

### EXAMPLES

The following Examples are included solely to aid in a more complete understanding of the subject invention. The Examples do not limit the scope of the invention described herein in any fashion.

### Example 1

The first example shows a testing methodology of anthocyanins in bilberry-infused extract. Here, the BIO-SHIELD technology works when absorption of high concentrates into this shield result in substantial yield of ELDER-MAX (an elderberry extract nutraceutical) from a product claim of 18% to 24-26% test result.

First, a bilberry-infused extract solution was prepared. 100 mg of bilberry-infused extract was then dissolved by adding 10 ml boiling water. The extract was heated in the boiling water for 30 minutes. The solution was then filtered, and distilled water was added to increase the volume to 50 ml which was used as the solution to be tested.

For the testing step, between 2-4 ml of the above solution of bilbery-infused extract was added to 10 ml acid alcohol. The solution was shaken for 30 minutes. The O.D. was determined using a UV-VIS spectrophotometer (Beckman, Inc. U.S.A.) at 535 nm using 1 cm colormeter cup spectrophotometer. Any precipitate in the solution, required centrifugation under 2000-2500 rpm for 5 minutes. The content of anthocyanidins was then calculated based on these spectrophotometer readings. The BIO-SHIELD method resulted in a substantial yield of ELDER-MAX between 24-26% absorption, while a control indicated only an 18% absorption.

### Example 2

Bioavailability of Actives from BIO-SHIELD: An In Vivo Study

The objectives of this study were to determine the sustained release and bioavailability of actives from the BIO-SHIELD delivery system in humans. The results presented here are from a preliminary experiment using two healthy volunteers. Caplets of CRAN-MAX (a cranberry-based nutraceutical used primarily for the treatment of urinary tract infections) was used for the study. Since the proanthocyanidins are the main active components in preventing urinary tract infection in cranberries, urinary metabolites of proanthocyanidins were monitored in the study. The biologically active proanthocyanidins in cranberry are mainly dimers and trimers consisting of predominantly epicatechin units. The polymers may undergo acid hydrolysis in the stomach and/or also catabolized by human colonic microflora into low molecular weight phenolic acids such as phenylacetic, phenylpropionic and phenylvaleric acids.

The volunteers were on a restricted diet 24 hr prior to the experiment. On the day of sampling, the volunteers ingested one 500 mg capsule of CRAN-MAX in the morning. The urine samples were collected at 2, 4, and 7 hr after consumption of the capsule. The volunteers were fasting until the 7 th hour sample was collected. The urine samples were stored at −20° C. before HPLC analysis. Before the BPLC analysis, the urine samples were concentrated using Sep-Pak C18 cartridge; 30 ml of urine was loaded on the cartridge and washed with 20 ml of water and the absorbed phenolic compounds were eluted with 2 ml of methanol. A known volume of the concentrated extract was subjected to acid hydrolysis using 2M HCI in aqueous methanol at 90° C. for 2 hr. Samples were analyzed by BPLC before and after hydrolysis, to determine the free and conjugated metabolites.

The HPLC system consisted of a Hitachi diode array detector L-4500, Hitachi L7100 pump and Rheodyne injector. The column was Phenomenex Prodigy, C-18, 5μ, 250×4.6 mm. The free polyphenols were analyzed using 5% formic acid (A) and Acetonitrile (B) using the gradient: 0-5 min, 10%B, 5-10 min, 10-15% B, 10-20 min 18% B, 20-25 min, 18-30% B, 25-30 min, 50% B, 30-50 min, 100% B. The flow rate was 1 ml/min and the absorbance was monitored at 280 nm. The hydrolyzed sample was analyzed using 0.1% phosphoric acid:acetonitrile:tetrahydrofuran (86:12.5:1.5) as the mobile phase.

The HPLC profiles indicate an increase in the number and concentration of several peaks over the seven hour period. Some of these peaks could be metabolites of proanthocyanidins. A peak in excretion was not noticed at the time period tested, indicating a sustained release of the compounds. Previous studies on anthocyanins and green tea polyphenols have indicated that the maximum urinary excretion occurs at 3-5 hrs after ingestion. Further work needs to be done to identify the metabolites and determine the time for maximum excretion by extending the period of evaluation.

It is understood that the invention is not confined to the particular construction and arrangement of parts herein illustrated and described, but embraces such modified forms thereof as come within the scope of the claims.

### CITIONS

Fossen, T et al., Colour and stability of pure anthocyanins influenced by pH including the alkaline region, Food Chem., 63:435-440 (1998).

Lapidot, T et al., Bioavailability of red wine anthocyanins as detected in human urine, Agric. Food Chem., 46:4297-4302 (1998).

Tsuda, T et al., Absorption and metabolism of cyanidin 3-O-beta-D-glucoside in rats, FEBS Lett., 449:179-182, (1999).

Tsuda, T et al., The role of anthocyanins as an antioxidant under oxidative stress in rats, Biofactors, 13: 133-139 (2000).

Youdim, K A et al., Polyphenolics enhance red blood cell resistance to oxidative stress: in vitro and in vivo, Biochem. Biophys. Acta-Gen. Subj., 1523:117-122 (2000).

What is claimed is:

1. A method of orally delivering nutraceutical products into the gastrointestinal tract of an animal for absorption, comprising:
a. expressing the juice from one or more plant materials comprising nutraceutical products to yield a juice portion and a pomace portion,
b. concentrating the juice portion to form a juice concentrate,
c. infusing the concentrate with the pomace portion, whereby the concentrate is absorbed into the pomace, and
d. orally delivering the product of step d to the animal.

2. The method of claim 1, wherein the pomace portion comprises a matrix suitable to protect the concentrate from digestive enzymes.

3. The method of claim 1, wherein the plant material is selected from the group consisting of fruits, vegetables, herbs and spices.

4. The method of claim 2, wherein the plant material is selected from seeds.

5. The method of claim 1, wherein the product of step c. is dried, milled and tableted or capsulated.

6. The method of claim 1, wherein the plant materials are is selected from the group consisting of cranberries, blueberries, bilberries, aronia, raspberries, Nigella sativa, saw palmetto, alfalfa, Echinacea, apples, carrots and squash.

7. The method of claim 1, wherein the pomace is cranberry pomace.

8. A method for passing intact anthocyanins into the lower gastrointestinal tract of a body for absorption from an aqueous medium comprising:
a. expressing juice from plant material, which juice include anthocyanins, thereby yielding a juice portion and a pomace portion;
b. concentrating the juice portion to yield a juice concentrate;
c. mixing the juice concentrate with the pomace portion in an about 1:1 to 1:4 (wt/wt) juice concentrate to pomace ratio to yield juice-infused pomace;
d. drying the juice-infused pomace to yield a free-flowing, non-hygroscopic powder formulation to yield the dietary supplement; and
e. orally ingesting the dietary supplement.

9. The method according to claim 8, wherein in step a., juice is expressed from one or more fruits selected from the group consisting of blueberries, aronia, bilberries, and raspberries.

10. The method according to claim 7, wherein in step a., juice is expressed from cranberries.

11. A dietary fiber product for use as a natural means of carrying bio-active ingredients produced according to steps a.-d. of claim 1.

12. The dietary fiber product of claim 11, wherein the plants materials are selected from the group consisting of cranberries, blueberries, bilberries, aronia, raspberries, Nigella saliva, saw palmetto, alfalfa, echinacea, apples, carrots and squash.

13. The dietary fiber product of claim 11, where plant material of the pomace is different from the plant material of the concentrate.

14. A method of producing a nutraceutical product comprising:
a. expressing juice from one or more fruits thereby yielding a juice portion and a pomace portion;
b. concentrating the juice portion to yield a juice concentrate; and
c. mixing the juice concentrate with the pomace portion to yield a juice-infused product.

15. The method of claim 14 wherein the juice concentrate is mixed with the pomace portion in an about 1:1 to 1:4 (wt/wt) juice concentrate to pomace ratio.

16. A drink product produced by the method of claim 14.

17. A food additive for fortification of fiber bars, cereals, breads, and drinks produced by the method of claim 14.
